(19) United States
(12) Patent Application Publication
(10) Pub. No.: US 2004/0172081 A1
(43) Pub. Date: Sep. 2, 2004

(54) Intracardiac pressure guided pacemaker

(76) Inventor: Dai-Yuan Wang, Bethesda, MD (US)

Correspondence Address: Law Offices of Albert Wai-Kit Chan, World Plaza, Suite 604, 141-07 20th Avenue, Whitestone, NY, 11357, US

(21) Appl. No.: 10/787,635

(22) Filed: Feb. 26, 2004

Related U.S. Application Data
(60) Provisional application No. 60/450,752, filed on Feb. 28, 2003.
(60) Provisional application No. 60/468,477, filed on May 7, 2003.

(51) Int. Cl. A61N 1/365

(52) U.S. Cl. 607/17

(57) ABSTRACT

The current invention is an intracardiac pressure guided pacemaker. It has a sensor in pacemaker lead to sense intracardiac pressure, which is used to find the best pacing location in the cardiac chamber and to adjust the timing of pacing signal. It will optimize pacing location and pacing timing and, therefore, optimize resychronization of the contraction of myocardium and interaction between different cardiac chambers. This will improve cardiac function at the same working condition without increase in oxygen demand from myocardium. The pacemaker has a computer program, which, based on intracardiac pressure, can adjust pacing parameters of the pacemaker to optimize resynchronization of the myocardium and interaction between different cardiac chambers automatically at different heart rate and in certain time interval specified by care provider. This also can be achieved by using an interrogator manually. The pacemaker can also store profiles of pacing parameter and intracardiac pressure, which can be retrieved for review to help optimize cardiac performance.

This application claims benefit of U.S. Serial No. 60/450,752, filed Feb. 28, 2003 and U.S. Serial No. 60/468,477, filed May 7, 2004, which are incorporated into this application by reference.

### BACKGROUND OF THE INVENTION

It is known that dysynchronization of contraction of the myocardium or between different cardiac chambers causes the heart to work inefficiently. This may cause decrease in cardiac output and congestive heart failure. One way to treat this condition is ventricular or biventricular pacing, which will resynchronize contraction of ventricular muscle and activity of the atria and ventricles by using electrical stimuli from pulse generator to pace the chamber or chambers of the heart. Usually this will be achieved by adjusting the timing of pacing signals to the atria and ventricle or position pacer leads in the cardiac chambers. Some methods have been developed to adjust the timing of pacing signals. Since the purpose of biventricular pacing is to resynchronize the activities of cardiac muscle and cardiac chambers and it translates into an increase in cardiac function, meaning increased ventricular performance (especially left ventricle) compared on a similar working load condition to the heart. Adjustment of pacing timing is an indirect method and may not always lead to optimal improvement of cardiac function and with improvement of cardiac function, those pacing timings may need to be readjusted. Even in the same condition of the heart, it may need to be adjusted at different heart rate. Multiple clinic visits are needed and optimal timing for the visits may be difficult to determine since each patient may respond to the pacing differently. This will mean sub-optimal patient care.

### SUMMARY OF INVENTION

If intracardiac pressure can be measured to guide the adjustment of the pacing setting, it will optimize pacing setting by finding optimal pacing location and timing. Therefore, resynchronization of the contraction of myocardium and interaction between different cardiac chambers will optimize resulting in improvement of function the heart without increase working load on the heart. Resynchronization may also shorten systole at given heart rate, which will in turn prolong diastole. This will improve perfusion of the myocardium since the perfusion of the myocardium mostly occurs during diastole.

The current invention consists of pacemaker and a pacemaker catheter, or catheters. The pacemaker catheter contains pacing leads and a sensor in its' distal end of pacing catheter for sensing intracardiac pressure or a separate catheter for sensing arterial blood pressure. Intracardiac pressure measured in a real time manner by the sensor is fed back to pacemaker and used to help position the pacing leads in the cardiac chamber or chambers and to optimize the pacing setting, which result in optimization of resynchronization of myocardium and interaction among different cardiac chambers. Adjustment of pacing setting of the pacemaker can be achieved manually by a care provider or automatically by the pacemaker containing a program for this purpose. The pacemaker can sense the intracardiac pressure in a real time manner and adjust pacing setting to optimize cardiac performance. This can be done at different heart rate. It can also be performed automatically at certain time interval specified by care provider. This will further optimize resynchronization of myocardium and interaction among different cardiac chambers and, therefore, improve the function of the heart. This adjustment can be achieved manually by care provider using an interrogator. The pacemaker has capability to store information about pacing, intracardiac pressure and any adjustment the data can be retrieved for further analysis.

A pacing lead inserted into left ventricle raises a concern that it may cause thrombosis or infection. Alternatively, a sensor may used to sense systemic blood pressure or rate of expansion of the artery. Time difference between QRS complex from intracardiac electrocardiogram or ventricular pacing signal and maximal dp/dt of arterial blood pressure or maximal rate of expansion the artery during cardiac cycle may be used as a segregate dp/dt intracardiac pressure since shorter the time difference between QRS complex or ventricular pacing signal and dp/dt or faster the rate of expansion of the artery, the faster increase in intraventricular pressure when heart rate and contractility of the ventricle remain unchanged, meaning the ventricle needs less time to built up pressure to open the aortic valve against diastolic arterial blood pressure. Since the heart is paced at programmed rate and other factors such as arterial blood pressure, peripheral resistance and circulation volume will remain unchanged. The time difference between maximal dp/dt of arterial pressure or the fastest expansion rate of the artery and QRS complex or ventricular pacing signal will depends on dp/dt of intraventricular pressure during early diastole. The higher the maximal intraventicular dp/dt, the better the performance of the ventricle. When the ventricle is paced, A-V delay and right/left ventricular synchronization can be optimized based on time difference between intracardiac electrocardiogram or ventricular pacing signal and maximal dp/dt of the arterial blood pressure, or simply based on maximal dp/dt of the arterial blood pressure or combination of both. A sensor can be used to sense arterial blood pressure at the subclavian or axillary artery, which can be approached during insertion of the pacemaker with slight modification of the procedure of the insertion of the pacemaker. Pacing location, A-V delay, and synchronization between left and right ventricles will be adjusted based on optimal timing difference between the QRS complex of intracardiac electrocardiogram and/or maximal dp/dt of systemic blood pressure by the sensor(s) placed on the subclavical artery or other arteries. The pacemaker can sense the arterial blood pressure in a real time manner and adjust pacing setting to optimize cardiac performance. This can be done at different heart rate different workload of the heart. It can also be performed automatically at certain time interval specified by care provider. This will further optimize resynchronization of myocardium and interaction among different cardiac chambers and, therefore, improve the function of the heart. This adjustment can be achieved manually by care provider using an interrogator. The pacemaker has capability to store information about pacing, arterial pressure, intracardiac electrocardiogram and any adjustment. The data can be retrieved for further analysis. To achieve this goal, a separate sensing catheter will connect a sensor from the artery(s) to the pacemaker. If biventricular pacing is used, sensing capability for intracardiac electrocardiogram and pressure of the left ventricular lead may be eliminated. It will decrease size of the catheter, which may make it safer and easier, especially when being introduced into cardiac venous system.

The pacemaker can be designed to use with one pacing catheter or multiple pacing catheters. This will allow incorporation multi-chamber pacing in one pacemaker.

### DETAIL DESCRIPTION OF THE FIGURES

FIG. 1. is a diagram of the pacing system. 2 is the pacemaker, which can receive data from a sensor in sensor and pacing lead or leads. It also can send electrical pulse to stimulate the myocardium through the pacing catheter. It contains computer programs to adjust pacing parameters based on intracardiac pressure measured by a sensor to achieve optimal cardiac performance. 4 is the pacing catheter for right atrium, which is anchored to myocardium of the right atrium and may contain pacing leads and a sensor in its' distal end. 6 is the right atrium of a human heart. 8 is the right ventricle of a human heart. The pacing catheter 10 is anchored in the apex of the right ventricle. The pacing leads can be inserted into any cardiac chamber or chambers as needed and connected to the same or different pacemaker. 12 is a human heart. 14 is the left ventricle. 16 is left atrium. 18 is a catheter containing a sensor in its' distal end, which is connected to an artery 22. 20 is a pacing catheter inserted into cavity of the left ventricle or into cardiac venous system for epicardial pacing.

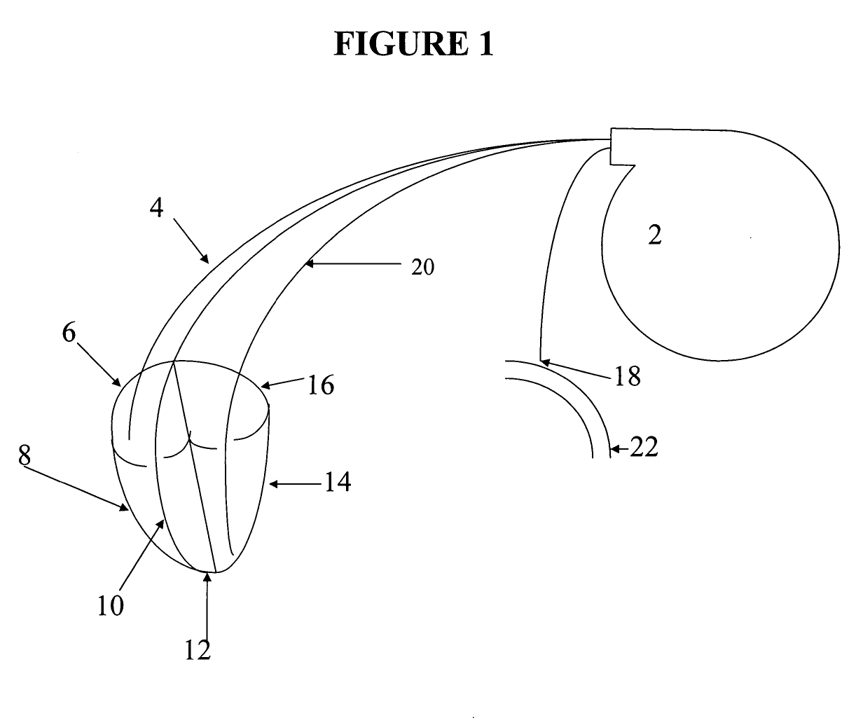

FIG. 2. is a diagram of arterial blood pressure and intracardiac electrocardiogram. FIG. 2 shows arterial pressure and intracardiac electrocardiogram tracings. 24 is a pacing spike. 26 is time interval between pacing spike and dp/dt of arterial blood pressure. 28 is dp/dt of arterial blood pressure. 30 is a tracing of arterial blood pressure. 32 is a tracing of intracardiac electrocardiogram. 34 is electrical activity of recorded cardiac chamber. 36 is time interval between intracardiac electrocardiogram and dp/dt of arterial blood pressure. dt is A-V delay in ms.

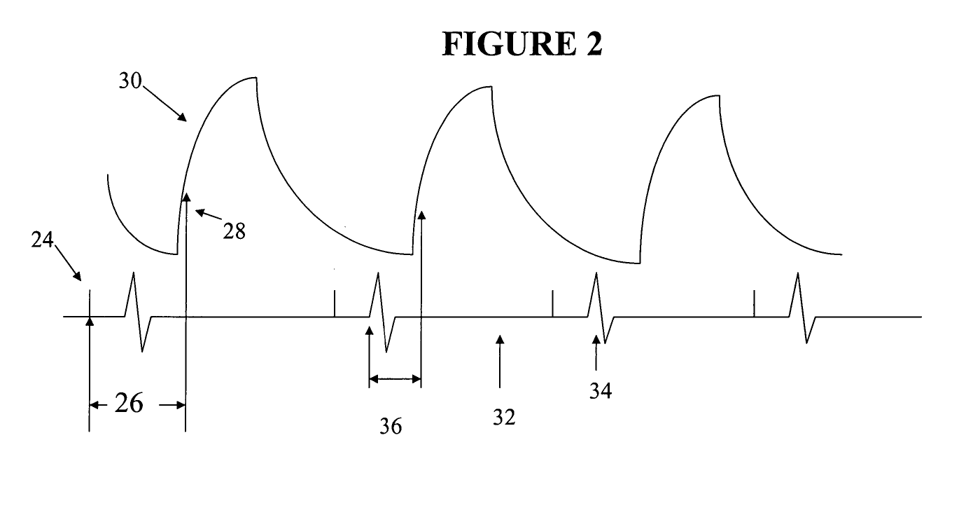

FIG. 3 is a flow chart of intracardiac pressure guided pacing protocol. R is pacing rate (beat per minute, bpm). Max dp/dt is maximal pressure change in the left ventricle during systole Lookup table contains optimal A-V delay at given pacing rate. Initial A-V delay may be shorter than 350 ms at higher pacing rate. Pacing rate (R′) will increase at 10 bpm increment from 50 bpm up to 120 bpm or otherwise specified. dt is A-V delay in ms.

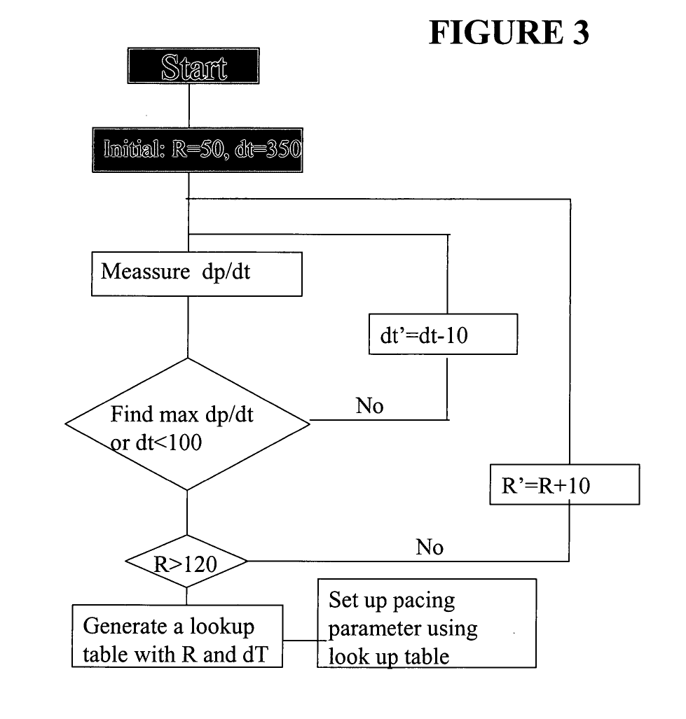

FIG. 4 is a flow chart of arterial pressure guided pacing protocol. R is pacing rate (beat per minute, bpm). dT is time interval from pacing signal to maximal dp/dt of arterial pressure. Lookup table contains optimal A/V delay at given pacing rate. Initial A-V delay may be shorter than 350 ms at higher pacing rate. dT may be replaced by maximal dp/dt when intraventricular pressure is used to find optimal A-V delay. Pacing rate (R′) will increase at 10 bpm increment from 50 bpm up 120 bpmor otherwise specified.

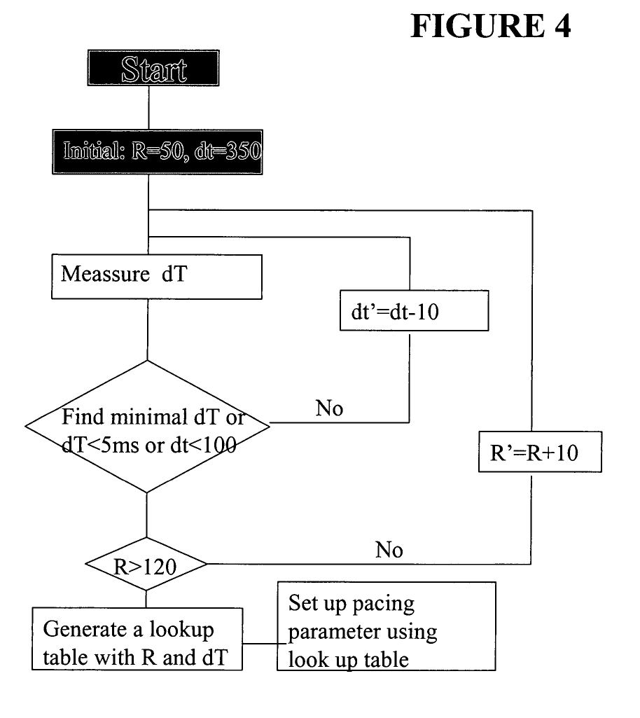

### DETAILED DESCRIPTION OF THE INVENTION

This invention provides a pacing system for pacing a heart, which can directly or indirectly sense intracardiac pressure of the human heart and pace the cardiac chamber of the heart based on intracardiac pressure or pressure change, said system comprises a pacing generator, a pacing catheter or catheters, pressure sensing catheter and a pacemaker interrogator.

In an embodiment, the pacing system comprises a pressure sensing catheter, which can sense arterial blood pressure and feed it back to said pacing generator.

In another embodiment, the pacing catheter comprises pacing lead or leads to deliver pacing signal to stimulate cardiac muscle to contract and a sensor to sense intracardiac pressure and pressure change.

In a further embodiment, the intracardiac pressure and pressure change may be replaced with blood pressure change in an artery or rate of expansion of an artery.

In a separate embodiment, of the above pacing system the pacemaker generator contains computer program or programs and is capable of receiving data from the sensor or sensors from pressure sensing catheter or intracardiac pacing catheter containing a sensor and intracardiac electrocardiogram from intracardiac catheter and to utilize the information to find optimal position of pacemaker lead or leads and optimal timing for delivery of pacing signal or signals.

In a further embodiment, of the pacing system the pacing lead contains one or multiple leads to deliver pacing signal or signals to stimulate the heart to cause the heart to contract, sense electrically activity of the heart and transmit those data to the pacemaker.

In an embodiment, the pacing generator contains a computer program to allow the generator to receive pressure and pressure change in the cardiac chamber or chambers from a sensor in said pacing catheter and, based on pressure and/or pressure change, to adjust pacing parameters delivered through the pacing lead or leads to optimize function of the heart.

In a further embodiment, the adjustment of pacing parameters is performed periodically and automatically by said pacing generator.

In a separate embodiment, the pacing generator will store information automatically, such as pacing parameters, activities of the heart, intracardiac pressure profile or profiles, and adjustment made by the pacing generator. In a further embodiment, the stored information is retrieved with a pacemaker interrogator for analysis and for adjustment of said pacing parameters through the interrogator.

In another embodiment, the pacing generator is connected to more than one pacing catheter for pacing the cardiac chamber at different locations or for pacing different cardiac chambers to achieve optimal performance of the heart.

The invention will be better understood by reference to the Example which follow, but those skilled in the art will readily appreciate that the specific experiments detailed are only illustrative, and are not meant to limit the invention as described herein, which is defined by the claims which follow thereafter.

### EXAMPLE

When a patient needs cardiac pacing, pacemaker leads will be in inserted into a cardiac chamber, such as the left ventricle, the pacing lead will be positioned to different location to find out which pacing location produce optimal intraventricular pressure and rate of change in the intracardiac pressure, which is likely to be maximal pressure and highest dp/dt. This is myocardial synchronization. After location of the pacing lead is determined and if contraction of the paced chamber will have to coordinate with other cardiac chamber or chambers, timing of delivery of pacing signal will be optimized based on intracardiac pressure measurement in coordination with other cardiac chamber or chambers. When more than one chamber are paced, adjustments of pacing setting for different chambers will be based on optimal pressure in a chamber interested, which is usually the left ventricle.

The optimal setting may be different at different heart rate, which can be adjusted based on intracardiac pressure at different heart rate. The pacemaker will contain a program, which will allow it to monitor the performance of the heart by measuring intracardiac pressure and dp/dt and adjust pacing timing to optimize cardiac performance based on intracardiac pressure measurement. This can be done automatically at the time of implantation of the pacemaker and at a certain time interval after implantation of the pacemaker, specified by the care provider. This may further optimize cardiac function since the function of the heart may change after certain time of pacing. Current pacing system can be used in single or multiple chamber pacing.

For example, when atria and left ventricle are paced, A-V conduction delay of the pacemaker will be adjusted based on optimal ventricular performance, which is related to optimal left ventricular pressure rising during the systole of the left ventricle. This can be achieved by pacing the atrium and left ventricle at a constant rate for a few minutes at different A-V conduction delay. This can be performed at different heart rate to optimize cardiac performance since optimal A-V conduction delay may be different at deferent heart rate. When right ventricle and left ventricle are paced, it may require right being paced at different timing from the left ventricle, this will be adjusted based on optimal pressure or dp/dt in the left ventricle. This also can be done at different heart rate.

This can be performed automatically by the pacemaker or manually by a care provider through an interrogator since any change in cardiac function may need readjustment of above pacing parameters. This is not limited to left ventricle. If right ventricular function needs to be optimized, the adjustment will be based on right ventricle.

Alternatively, a pacing lead for pacing left ventricle can be inserted into the coronary sinus, even further advanced to the apex of the heart through a vein, such as middle cardiac vein. A sensor will be placed on the subclavian or axillary artery to sense pressure change. Time difference between a pacing signal or intracardiac electrocardiogram and dp/dt in blood pressure, which follows the ventricular contraction, will be used to locate best pacing location and, then, best pacing timing as described with intraventricular pressure guided adjustments in previous paragraph. The shortest or nearly shortest time from pacing signal or QRS complex of intracardiac electrocardiogram indicates that the pacing location or timing produce best synchronization. Placement of a sensor on the artery can be done during insertion of pacemaker with slight modification of routine pacemaker insertion since routine sites for pacemaker insertion is very close to the subclavian or axillary arteries. The sensor will be attached to the surface of the artery with minimal pressure to avoid interference to function of artery or damage to the artery.

What is claimed is:

1. A pacing system for pacing a heart, which can directly or indirectly sense intracardiac pressure of the heart and pace the cardiac chamber of the heart based on intracardiac pressure or pressure change, said system comprises a pacing generator, at least one pacing catheter, pressure sensing catheter and a pacemaker interrogator.

2. The pacing system of claim 1, comprising a sensing catheter, which can sense arterial blood pressure and feed it back to said pacing generator.

3. The pacing system of claim 1, wherein the pacing catheter comprises pacing lead or leads to deliver pacing signal to stimulate cardiac muscle to contract and a sensor to sense intracardiac pressure and pressure change.

4. The pacing system of claim 3, wherein the intracardiac pressure and pressure change is replaced with blood pressure change in an artery or rate of expansion of an artery.

5. The pacing system of claim 1, wherein said pacemaker generator contains computer program or programs and is capable of receiving data from the sensor or sensors from pressure sensing catheter or intracardiac pacing catheter containing a sensor and intracardiac electrocardiogram from intracardiac catheter and to utilize the information to find optimal position of pacemaker lead or leads and optimal timing for delivery of pacing signal or signals.

6. The pacing system of claim 5, wherein said pacing lead contains one or multiple leads to deliver pacing signal or signals to stimulate the heart to cause the heart to contract, sense electrically activity of the heart and transmit those data to the pacemaker.

7. The pacing system of claim 1, wherein said pacing generator contains a computer program to allow the generator to receive pressure and pressure change in the cardiac chamber or chambers from the sensor in said pacing catheter and, based on pressure and/or pressure change, to adjust pacing parameters delivered through the pacing lead or leads to optimize function of the heart.

8. The pacing system of claim 7, wherein said adjustment of pacing parameters is performed periodically and automatically by said pacing generator.

9. The pacing system of claim 1, wherein said pacing generator will store information automatically, such as pacing parameters, activities of the heart, intracardiac pressure profile or profiles, and adjustment made by the pacing generator.

10. The pacing system of claim 9, wherein said store information is retrieved with a pacemaker interrogator for analysis and for adjustment of said pacing parameters through the interrogator.

11. The pacing system of claim 1, wherein said pacigenerator is connected to more than one pacing catheter for pacing the cardiac chamber at different locations or for pacing different cardiac chambers to achieve optimal performance of the heart.

12. The pacing system of claim 1, wherein the heart is a human heart.
